(19) United States
(12) Patent Application Publication
(10) Pub. No.: US 2020/0222513 A1
(43) Pub. Date: Jul. 16, 2020

(54) BOTULINUM TOXIN FOR SPASMOLYSIS IN REVASCULARIZATION

(71) Applicant: University of Southern California, Los Angeles, CA (US)

(72) Inventors: Jonathan Russin, Los Angeles, CA (US); Sebina Bulic, Los Angeles, CA (US)

(73) Assignee: University of Southern California, Los Angeles, CA (US)

(21) Appl. No.: 16/743,881

(22) Filed: Jan. 15, 2020

Related U.S. Application Data
(60) Provisional application No. 62/792,598, filed on Jan. 15, 2019.

(51) Int. Cl. A61K 38/48; A61F 2/06; A61K 9/00

(52) U.S. Cl. CPC A61K 38/4893; A61F 2/06; A61F 2250/0067; A61F 2240/001; A61K 9/0024

(57) ABSTRACT

Graft spasm is a common complication of bypass procedures and can result in ischemia or graft thrombosis. Described herein is use of botulinum toxin to prevent graft spasm in bypass surgery. The technique was used in extracranial-intracranial (EC-IC) bypass surgeries, with the harvested graft treated ex vivo with botulinum toxin before the anastomosis was performed. Post-bypass vascular imaging demonstrated patency and the absence of spasm in grafts, without any immediate endothelial or vessel wall damage. Postoperative angiograms were without graft spasm in all cases. Botulinum toxin can therefore be used to prevent graft spasm and maintaining patency in cerebral revascularization procedures.

### FIELD OF THE INVENTION

Described herein are methods and compositions for preventing spasms in grafts used for human revascularization procedures.

### BACKGROUND

The maintenance of flow is critical to the success of bypass surgery, but can be complicated by the need for interposition grafts connecting donor and recipient vessels due to the risk of vessel spasm and resultant thrombosis and/or ischemia. This includes, for example, the extracranial-intracranial (EC-IC) bypass procedure—a valuable treatment modality for complex cerebral aneurysms and refractory symptomatic vessel occlusions.

While bypass grafts are typically selected based on flow capacity and size matching to recipient and donor vessels, the spasm risk and patency rates of different grafts are also considered. While neurosurgical, plastic surgery, and cardiovascular reports have identified strategies to decrease the risk of graft spasm and/or thrombosis. This includes maintaining an elevated mean arterial pressure and using vasodilators in the immediate post-operative period, devoting attention to atraumatic graft harvests, and using antiplatelet agents. Ex vivo graft treatments with short-acting vasodilators prior to implantation have also been described. Despite these advances, no definitive spasm prevention strategy exists. There is a great need in the art for preventing spasms and associated complications in vascular grafts.

Botulinum toxin (BTX) is a powerful neurotoxin used safely in a multitude of clinical settings for muscle relaxation. It has also been considered for the prevention of arterial graft spasm in preclinical cardiovascular and plastic surgery assessments, although no data for this application yet exists.

Described herein, the Inventors report on the first use of BTX to prevent graft spasm in 3 patients undergoing EC-IC bypass. The Inventors also demonstrate, via histopathological analysis, the absence of any immediate endothelial or vessel wall damage from the BTX treatment, thereby demonstrating effective use of botulinum toxin for the prevention of arterial graft spasm in human revascularization procedures.

### SUMMARY OF THE INVENTION

Described herein is a method, including incubating a vascular graft with an anti-spasm agent, transplanting the incubated vascular graft into a subject. In other embodiments, the anti-spasm agent is botulinum toxin (BTX). In other embodiments, the BTX is type A, B, C, D, E, F, G and combinations thereof. In other embodiments, the vascular graft is an autograft. In other embodiments, the vascular graft is an allograft. In other embodiments, the vascular graft is an artery or vein. In other embodiments, the artery is a radial artery or descending branch of the lateral circumflex femoral artery. In other embodiments, the vein is a saphenous vein. In other embodiments, transplanting the incubated vascular graft is for extracranial-intracranial bypass surgery. In other embodiments, the subject is administered a vasodilator.

Further described herein is a method of bypass surgery, including transplanting a vascular graft into a subject, wherein the vascular graft has been treated with an anti-spasm agent. In other embodiments, the anti-spasm agent is botulinum toxin (BTX). In other embodiments, the BTX is type A, B, C, D, E, F, G and combinations thereof. In other embodiments, treatment with BTX includes incubation for about 5 to 120 mins. In other embodiments, treatment with BTX includes 5-250 U in 5-30 ml of saline. In other embodiments, the bypass surgery is extracranial-intracranial bypass surgery. In other embodiments, the vascular graft is a radial artery, descending branch of the lateral circumflex femoral artery or saphenous vein.

Also described herein is a method of preparing a vascular graft, including incubating a vascular graft with botulinum toxin (BTX). In other embodiments, the BTX is type A, B, C, D, E, F, G and combinations thereof. In other embodiments, incubating the vascular graft with BTX is for about 5 to 120 mins. In other embodiments, incubating the vascular graft with BTX includes 5-250 U of BTX in 5-30 ml of saline.

Further described herein is a vascular graft made by a method including incubating a vascular graft with an anti-spasm agent, transplanting the incubated vascular graft into a subject. In other embodiments, the In other embodiments, the anti-spasm agent is botulinum toxin (BTX). In other embodiments, the BTX is type A, B, C, D, E, F, G and combinations thereof. In other embodiments, the vascular graft is an autograft. In other embodiments, the vascular graft is an allograft. In other embodiments, the vascular graft is an artery or vein. In other embodiments, the artery is a radial artery or descending branch of the lateral circumflex femoral artery. In other embodiments, the vein is a saphenous vein. In other embodiments, transplanting the incubated vascular graft is for extracranial-intracranial bypass surgery. In other embodiments, the subject is administered a vasodilator.

### BRIEF DESCRIPTION OF THE FIGURES

FIG. 1. Case 1. Preoperative workup demonstrated left ICA occlusion with significant penumbral tissue. Lateral (A) and oblique (B) angiograms obtained in a 57-year-old man presenting with acute aphasia and hemiparesis, demonstrating a cervical left ICA occlusion. A CT angiography study demonstrated vessel reconstitution beyond the ophthalmic segment. Axial diffusion-weighted (left, C) and perfusion-weighted (right, C) Mill studies demonstrated scattered anterior cerebral artery and MCA infarcts with a large ischemic penumbra in the left MCA distribution, as seen by (from left to right) increased mean transit times, decreased cerebral blood flow, and preserved cerebral blood volumes.

FIG. 2. Case 1. Postoperative imaging and histology demonstrated EC-IC graft health and the absence of spasm following ex vivo BTX treatment. Postoperative day 2 anteroposterior (A) and lateral (B) angiograms demonstrating a patent STA-MCA interposition graft (arrows) without evidence of spasm. Intrinsic left ICA flow is improved in these images because of partial endovascular treatment of the occlusion. A photomicrograph (C) of a portion of the DLCFA graft following BTX treatment demonstrated an intact endothelium and vessel muscular walls, with no structural deformities. H & E, original magnification ×100.

FIG. 3. Case 2. Preoperative vessel imaging demonstrated a large basilar tip aneurysm. CT angiography study obtained in a 45-year-old man following an SAH, demonstrating a broad-based, bilobed basilar tip aneurysm, with the patient's anterior circulation entirely dependent on the posterior communicating arteries. The subject was treated with an STA-MCA bypass with a BTX-treated DLCFA graft for revascularization of the anterior circulation in conjunction with clip ligation of the basilar tip aneurysm.

FIG. 4. Case 2. Postoperative angiography and histology demonstrated EC-IC graft health and the absence of spasm following BTX treatment. Postoperative day 11 anteroposterior (A) and lateral (B) angiograms demonstrating a patent STA-MCA with a DLCFA graft (arrows) that had been treated ex vivo with BTX. *Site of spasm on untreated distal STA at the clip site. Beginning of graft. A comparison of low-magnification and high-magnification images of untreated (C and D, respectively) and BTX-treated (E and F, respectively) portions of the DLCFA graft demonstrates integrity of the endothelium and vessel wall and no structural deformities after BTX treatment. Vasodilation was also noted in the treated sample. H & E, original magnification □40 (C and E), ×100 (D and F).

FIG. 5. Case 3. Postoperative angiography and histology in a 56-year-old man treated using an STA-MCA bypass with a BTX-treated DLCFA graft for a progressively symptomatic, pressure-dependent right ICA occlusion. Day 4 postoperative anteroposterior (A) and lateral (B) angiograms demonstrated no spasm and graft patency. Arrows indicate the bypass graft. Site of STA-graft anastomosis. Low-magnification (C) and high-magnification (D) images of a portion of the BTX-treated DLCFA demonstrated no evidence of endothelial or vessel wall injury. H & E, original magnification ×40 (C), ×100 (D).

FIG. 6. Angiograms (A and B) from previous cerebral bypass cases using DLCFA grafts, which were harvested in a manner similar to that in the featured cases, but without BTX treatment, demonstrating a susceptibility for postoperative vasospasm (arrows). When such vasospasm occurred, multiple rounds of intraarterial verapamil injections were typically needed for management.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul., 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Abbreviations used herein including the following: BTX=botulinum toxin; DLCFA=descending branch of the lateral circumflex femoral artery; EC-IC=extracranial-intracranial; ICA=internal carotid artery; MCA=middle cerebral artery; RA=radial artery; SAH=subarachnoid hemorrhage; STA=superficial temporal artery.

Surgical revascularization continues to play an important role in the management of complex intracranial aneurysms and ischemic cerebrovascular disease. Graft spasm is a common complication of bypass procedures and can result in ischemia or graft thrombosis. Described herein is the use of botulinum toxin to prevent graft spasm following bypass surgery, specifically extracranial-intracranial (EC-IC) bypass.

This technique was used in 3 EC-IC bypass surgeries, 2 for symptomatic carotid artery occlusions and 1 for a ruptured basilar tip aneurysm. In all 3 cases, the harvested graft was treated ex vivo with botulinum toxin before the anastomosis was performed. Post-bypass vascular imaging demonstrated patency and the absence of spasm in all grafts. Histopathological analyses of treated vessels did not show any immediate endothelial or vessel wall damage. Postoperative angiograms were without graft spasm in all cases.

Based on the results, botulinum toxin is a reasonable option for preventing graft spasm and maintaining patency in cerebral revascularization procedures.

Described herein is a method, including incubating a vascular graft with an anti-spasm agent, transplanting the incubated vascular graft into a subject. In other embodiments, the anti-spasm agent is botulinum toxin (BTX). In other embodiments, the BTX is type A, B, C, D, E, F, G and combinations thereof. In other embodiments, the vascular graft is an autograft. In other embodiments, the vascular graft is an allograft. In other embodiments, the vascular graft is an artery or vein. In other embodiments, the artery is a radial artery or descending branch of the lateral circumflex femoral artery. In other embodiments, the vein is a saphenous vein. In other embodiments, vascular grafts include anterior or posterior tibial artery, superficial temporal artery, occipital artery, inferior epigastric artery, internal maxillary artery, external carotid artery, internal carotid artery, facial artery, arterial and venous grafts associated with free flaps. In other embodiments, arteries could be used in, for example, visceral revascularization, including celiac artery, superior mesenteric artery, inferior mesenteric artery, splenic artery, renal artery. In other embodiments, arteries could be used in, for example, coronary revascularization: coronary arteries, internal mamillary arteries, radial arteries. One of ordinary skill that a vascular graft for a particular application depends on relevant caliber and patency of the graft and the transplant site, with additional vascular graft sources available based on the anti-spasm techniques described herein. In other embodiments, transplanting the incubated vascular graft is for coronary artery bypass, free flap procedures, limb revascularization, visceral revascularization and extracranial-intracranial bypass surgery. In other embodiments, transplanting the incubated vascular graft is for extracranial-intracranial bypass surgery. In other embodiments, the subject is administered a vasodilator. In various embodiments, BTX could be used in combination with calcium channel blockers, phosphodiesterase inhibitors or nitroglycerin.

Described herein is a method of bypass surgery, including transplanting a vascular graft into a subject, wherein the vascular graft has been treated with an anti-spasm agent. In other embodiments, the anti-spasm agent is botulinum toxin (BTX). In other embodiments, the BTX is type A, B, C, D, E, F, G and combinations thereof. In other embodiments, the BTX is type A. In other embodiments, the BTX is type B. In other embodiments, treatment with BTX includes incubation for about 5 to 120 mins. In other embodiments, treatment with BTX includes 5-250 U in 5-30 ml of saline. In other embodiments, this includes 100 U in 10 mL saline for 30 minutes. In other embodiments, this includes 200 U in 10 mL saline for 30 minutes. In other embodiments, the bypass surgery is for aneurysm, atherosclerotic vessel occlusion, moyamoya angiopathy, traumatic vessel dissection, and major intracranial vessel involvement. In other embodiments, the bypass surgery is extracranial-intracranial bypass surgery. In other embodiments, the bypass surgery is coronary artery bypass. In other embodiments, the vascular graft is a radial artery, descending branch of the lateral circumflex femoral artery or saphenous vein. In other embodiments, vascular grafts include anterior or posterior tibial artery, superficial temporal artery, occipital artery, inferior epigastric artery, internal maxillary artery, external carotid artery, internal carotid artery, facial artery, arterial and venous grafts associated with free flaps. In other embodiments, arteries could be used in, for example, visceral revascularization, including celiac artery, superior mesenteric artery, inferior mesenteric artery, splenic artery, renal artery. In other embodiments, arteries could be used in, for example, coronary revascularization: coronary arteries, internal mamillary arteries, radial arteries. One of ordinary skill that a vascular graft for a particular application depends on relevant caliber and patency of the graft and the transplant site, with additional vascular graft sources available based on the anti-spasm techniques described herein. In other embodiments, transplanting the incubated vascular graft is for coronary artery bypass, free flap procedures, limb revascularization, visceral revascularization and extracranial-intracranial bypass surgery. In other embodiments, transplanting the incubated vascular graft is for extracranial-intracranial bypass surgery. In other embodiments, the subject is administered a vasodilator. In various embodiments, BTX could be used in combination with calcium channel blockers, phosphodiesterase inhibitors or nitroglycerin.

In other embodiments, the method includes transplanting a vascular graft into a subject for extracranial-intracranial bypass surgery on the subject, wherein the vascular graft has been treated with BTX type A by incubation with the BTX type A 100 U in 10 mL saline for 30 minutes. other embodiments, the method includes transplanting a vascular graft into a subject for extracranial-intracranial bypass surgery on the subject, wherein the vascular graft has been treated with BTX type A by incubation with the BTX type A 2 other embodiments, the method includes transplanting a vascular graft into a subject for extracranial-intracranial bypass surgery on the subject, wherein the vascular graft has been treated with BTX type A by incubation with the BTX type A 100 U in 10 mL saline for 30 minutes. 00 U in 10 mL saline for 30 minutes. In other embodiments, the vascular graft is a radial artery, descending branch of the lateral circumflex femoral artery, or saphenous vein.

Described herein is a method of preparing a vascular graft, including incubating a vascular graft with botulinum toxin (BTX). In other embodiments, the BTX is type A, B, C, D, E, F, G and combinations thereof. In other embodiments, the BTX is type A. In other embodiments, the BTX is type B. In other embodiments, incubating the vascular graft with BTX is for about 5 to 120 mins. In other embodiments, incubating the vascular graft with BTX includes 5-250 U of BTX in 5-30 ml of saline. In other embodiments, this includes 100 U in 10 mL saline for 30 minutes. In other embodiments, this includes 200 U in 10 mL saline for 30 minutes.

A vascular graft made by a method, including incubating a vascular graft with an anti-spasm agent, transplanting the incubated vascular graft into a subject. In other embodiments, the anti-spasm agent is botulinum toxin (BTX). In other embodiments, the BTX is type A, B, C, D, E, F, G and combinations thereof. In other embodiments, the BTX is type A. In other embodiments, the BTX is type B. In other embodiments, the vascular graft is an autograft. In other embodiments, the vascular graft is an allograft. In other embodiments, the vascular graft is an artery or vein. In other embodiments, the artery is a radial artery or descending branch of the lateral circumflex femoral artery. In other embodiments, the vein is a saphenous vein. In other embodiments, transplanting the incubated vascular graft is for extracranial-intracranial bypass surgery. In other embodiments, the subject is administered a vasodilator. In other embodiments, the method includes preparing a vascular graft, including incubating a vascular graft with botulinum toxin (BTX). In other embodiments, the BTX is type A, B, C, D, E, F, G and combinations thereof. In other embodiments, incubating the vascular graft with BTX is for about 5 to 120 mins. In other embodiments, incubating the vascular graft with BTX includes 5-250 U of BTX in 5-30 ml of saline. In other embodiments, this includes 100 U in 10 mL saline for 30 minutes. In other embodiments, this includes 200 U in 10 mL saline for 30 minutes.

### Example 1

### Methods

Retrospective analysis of an IRB-approved, prospectively maintained database was performed to identify patients who had undergone EC-IC cerebral bypass surgery using grafts treated with BTX. Recorded information included patient demographics (age, sex), clinical presentation, and surgery performed, as well as imaging and neurological outcomes.

All procedures for vessel harvest and donor site anastomosis were performed. Patients were considered for cerebral revascularization after conservative treatments had failed or if lesions were not amenable to traditional microsurgical or endovascular approaches. The revascularization strategy was planned through collaboration with the plastic surgery and neurosurgical teams. In all cases a descending branch of the lateral circumflex femoral artery (DLCFA) graft was used as the bypass vessel, which was treated ex vivo with BTX before implantation. A small section of each treated (and untreated in 1 patient) DLCFA graft was collected and sent for histopathological analysis via standard H & E staining.

After the patient's right thigh was prepped and draped, a dissection plane between the rectus femoris and vastus lateralis muscles was developed. The DLCFA was dissected for approximately 10 cm using 3.0 nylon ties to ligate tributaries. The graft was ligated proximally and distally and then cut sharply and removed from the leg. The adventitia was removed, and the graft was flushed and then soaked using 100 U of BTX type A (Allergan Inc.) in 10 ml of normal saline for approximately 30 minutes. Before the graft was mobilized to the intracranial space, it was flushed with a heparin and milrinone solution, a standard graft irrigation solution consisting of 10,000 U of heparin with 10 mg of milrinone in 1 L of normal saline.

### Example 2

### Results

Three patients, 2 for symptomatic carotid artery occlusions and 1 for a ruptured basilar tip aneurysm, had undergone superficial temporal artery (STA) to middle cerebral artery (MCA) EC-IC bypass surgery utilizing DLCFA grafts treated ex vivo with BTX prior to implantation. The average patient age was 52.6 years, and all 3 patients were male. The bypass procedure was technically successful in all cases. None of the patients exhibited imaging or clinical signs of postoperative graft spasm. Histopathological analysis of the treated vessels demonstrated no endothelial or vessel wall injury.

### Example 3

### Case 1

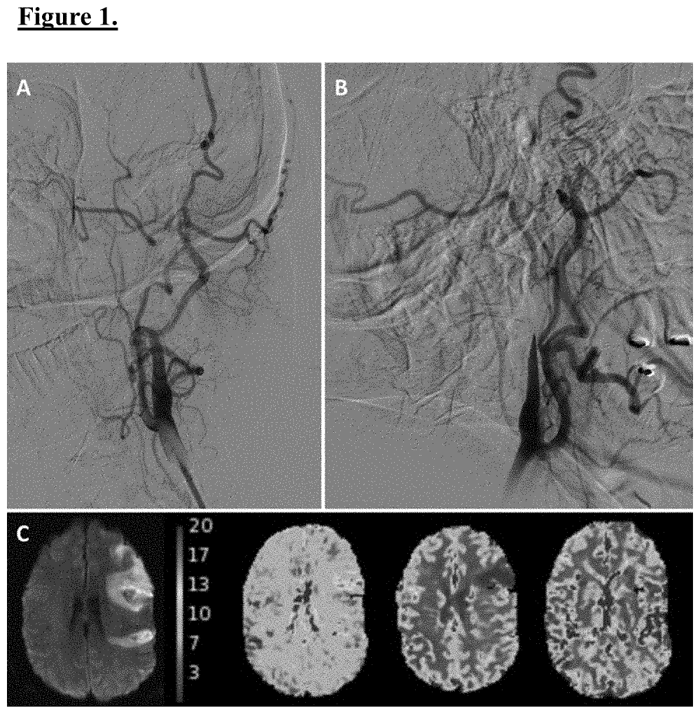

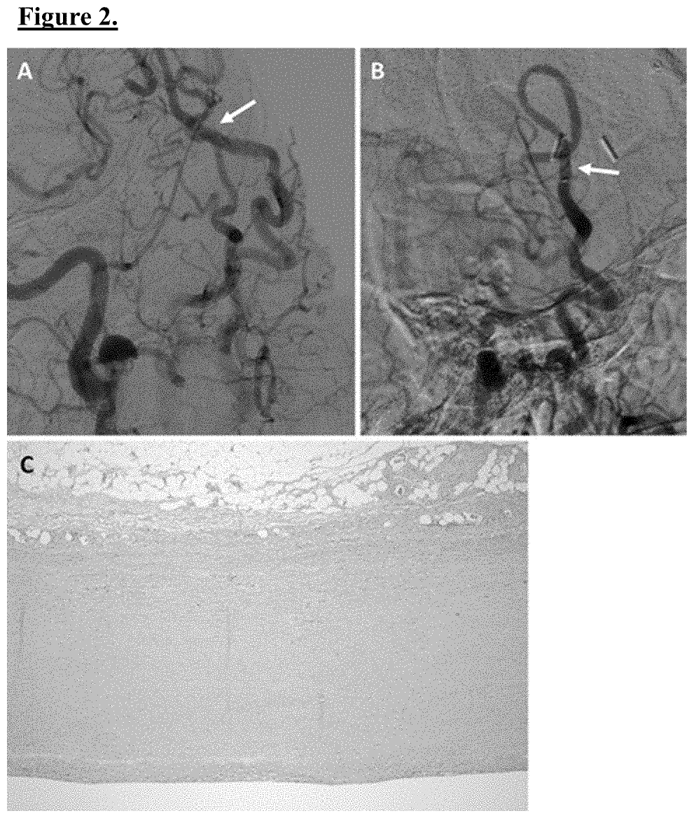

A 57-year-old man presented with the acute onset of dense mixed aphasia and right-sided paresis and was found to have a left internal carotid artery (ICA) occlusion. Pre-operative CT angiography demonstrated occlusion of the cervical portion of the ICA with reconstitution beyond the ophthalmic segment (FIGS. 1A and B). Perfusion-weighted MM demonstrated scattered anterior cerebral artery and MCA infarcts with a large ischemic penumbra incorporating the entire left MCA territory (FIG. 1C). An endovascular attempt to open the occluded vessel moderately improved ICA flow but was ultimately unsuccessful. An STA-MCA bypass with a BTX-treated DLCFA graft was performed. Postoperative Mill demonstrated no new ischemia, and angiography showed a patent graft without evidence of spasm (FIGS. 2A and B). Histopathological analysis revealed no immediate effects of BTX treatment on the endothelium or vessel wall (FIG. 2C). The patient had an unremarkable postoperative hospital course before discharge to a rehabilitation facility. By the 4.5-month follow-up, the subject was residing at home, his speech was con-versational, and his motor function had improved to being ambulatory without assistance (with stable right-sided upper extremity strength).

### Example 4

### Case 2

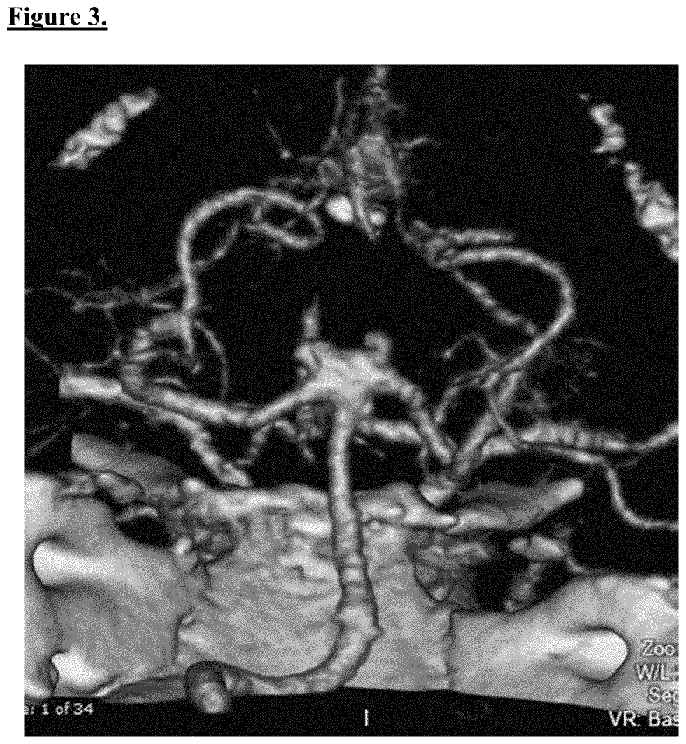

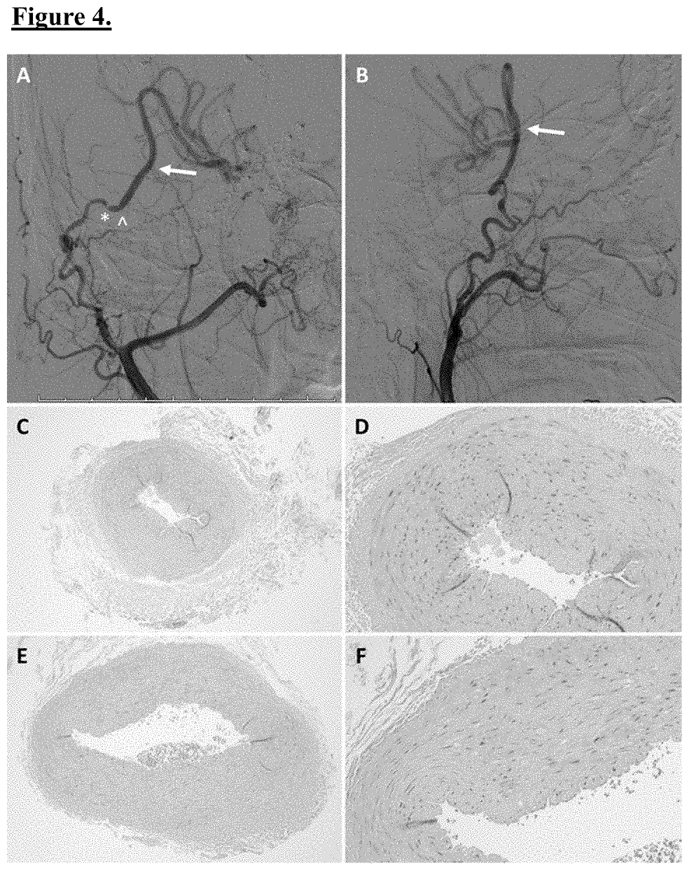

A 45-year-old man presented with the acute onset of headaches and was found to have a subarachnoid hemorrhage (SAH) from a ruptured, broad-based, bilobed basilar tip aneurysm (FIG. 3). Angiography demonstrated bilateral carotid arteries that terminated in the ophthalmic arteries, and the intracranial circulation was entirely dependent on the basilar artery. Given the complicated morphology of the basilar tip aneurysm, the Inventors anticipated that the subject would require temporary clipping of the basilar artery. The subject was, therefore, recommended for revascularization of the anterior circulation in conjunction with clip ligation of the basilar tip aneurysm. The subject underwent STA-MCA bypass with a BTX-treated DLCFA graft. Immediate postoperative CT angiography and postoperative day 11 angiography demonstrated a patent graft with no evidence of spasm (FIGS. 4A and B). Graft histopathology demonstrated no immediate adverse effects of BTX treatment on the endothelium or vessel wall (FIG. 4C-F). The patient had an unremarkable postoperative recovery and was transferred to a rehabilitation facility once medically cleared. By the 2-month follow-up, the subject was living at home and remained neurologically intact.

### Example 5

### Case 3

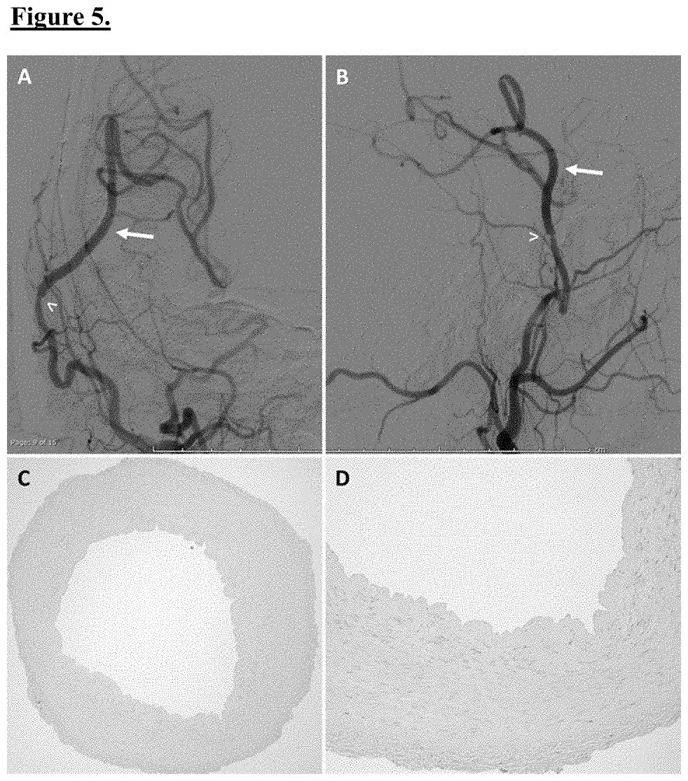

A 56-year-old man presented with acute left-sided weakness and was found to have a right ICA occlusion. His CT revealed perfusion deficits, and several attempts to wean him off of vasopressors failed with worsening leftsided weakness to the point of being barely antigravity. The subject underwent STA-MCA bypass with a BTX-treated DLCFA graft. His postoperative neurological exam was stable with blood pressure normalization. Postoperative CT angiography and conventional angiography demonstrated a patent bypass with no evidence of spasm (FIGS. 5A and B) and improved right-sided perfusion. Graft histopathology demonstrated no evidence of endothelial or vessel wall injury (FIGS. 5C and D). His remaining hospital course was unremarkable, and the subject had improving left-sided strength prior to discharge to a rehabilitation facility. By the 2-week follow-up, the subject was ambulatory in a rehabilitation facility with continued improvements in left-sided strength.

### Example 6

### Discussion

Ensuring vessel patency is critical to the success of graft-based EC-IC bypass. Graft spasm represents a particularly challenging pathology given its potential to rapidly and severely alter blood flow. Historically, the 2 main options for cerebral bypass grafts have been the radial artery (RA) and the saphenous vein. While there are advantages and disadvantages to both, RA grafts are generally preferred for EC-IC bypass given their higher overall patency rates and better donor-recipient vessel size matching. Nonetheless, RA grafts are at risk for spasm, an extreme smooth muscle-mediated vasoconstrictive response to mechanical or pharmacological stimuli, which can occur in up to 10% of cases. Other arterial grafts have also been described, such as the DLCFA graft that was used in the current series because of its closer size match to the donor STA, but are similarly susceptible to spasm.

When spasm occurs, treatment options include systemic anticoagulation, intraarterial injection of the calcium channel blocker verapamil and the antispasmodic papaverine, angioplasty, and local application of vasodilators. However, the prevention of graft spasm is the preferred strategy, and both mechanical and pharmacological prophylactic approaches have been described. As disruptions in the endothelium can lead to the release of spasmogenic agents, or spasmogens, such as endothelin and prostanoids, a meticulous surgical technique and preservation of the endothelium during graft harvest and implantation are important initial strategies for decreasing spasm risk. Preservation of the venae comitantes during harvest, along with both arterial and venous anastomoses, has also been suggested as a method of preserving the viability of tissues immediately surrounding the bypass graft, potentially decreasing spasm risk by reducing local oxidative stress.

Pharmacological prophylaxis of graft spasm is more robust in other surgical fields and typically involves treatment of the graft with a vasodilator prior to implantation, along with postoperative systemic infusion of vasodilators. Protocols from the cardiovascular literature include ex vivo treatment with a verapamil plus nitroglycerin solution, often followed by the systemic administration of calcium channel blockers with or without long-acting nitrates. Other topical pharmacological agents, such as the synthetic prostacyclin iloprost and diltiazem, have also been explored. However, the half-lives of these therapies are minutes to hours, and efficacy data on these techniques are limited. A uniform ex vivo treatment and postoperative protocol for EC-IC bypass graft spasm prevention does not currently exist.

In this setting, BTX has been suggested as a potentially long-term spasmolytic for arterial grafts. This irreversible toxin, produced by the anaerobic, gram-positive bacterium Clostridium botulinum, consists of 7 distinct serotypes (A-G), with types A and B most often used in the clinical setting. While the primary mechanism of action of all BTX subtypes is through presynaptic cleavage of SNARE (soluble NSF attachment protein receptor) proteins important for acetylcholine release into the synaptic terminal, the mechanism of its effects on arterial graft spasm is less clear, as neuronally mediated spasm of RA or DLCFA grafts is predominantly adrenergic. Secondary pathways are thus likely to contribute since the release of vasoconstricting catecholamines is also affected by BTX A-SNARE cleavage, and BTX A has been shown to inhibit the presynaptic release of vasoconstricting agents like substance P, as well as to increase the concentration of vasodilating calcitonin-related peptides. Botulinum toxin C has also been shown to block the GTP-dependent phosphorylation of myosin light chains in vascular smooth muscle, inhibiting constriction. These mechanistic effects are probably occurring in the setting of neuronal hyperactivity proposed to result from surgical denervation.

To date, assessments of BTX for graft spasm prevention have been limited to preclinical cardiovascular and plastic surgery studies. In these works, in vivo rat perivascular pretreatments with BTX B augmented microvessel diameter prior to anastomosis, and ex vivo treatment of rat aortas with BTX C resulted in the complete loss of adrenergic muscle contraction through the 2-hour study end point (significantly longer than the effect of papaverine). In vivo perivascular pretreatment with BTX A was also shown to increase vessel diameter and decrease short-term thrombosis rates in rat and rabbit micro-anastomosis models. The arterial wall was not affected by the BTX treatment in any of these studies, and all in vivo treatments were well tolerated.

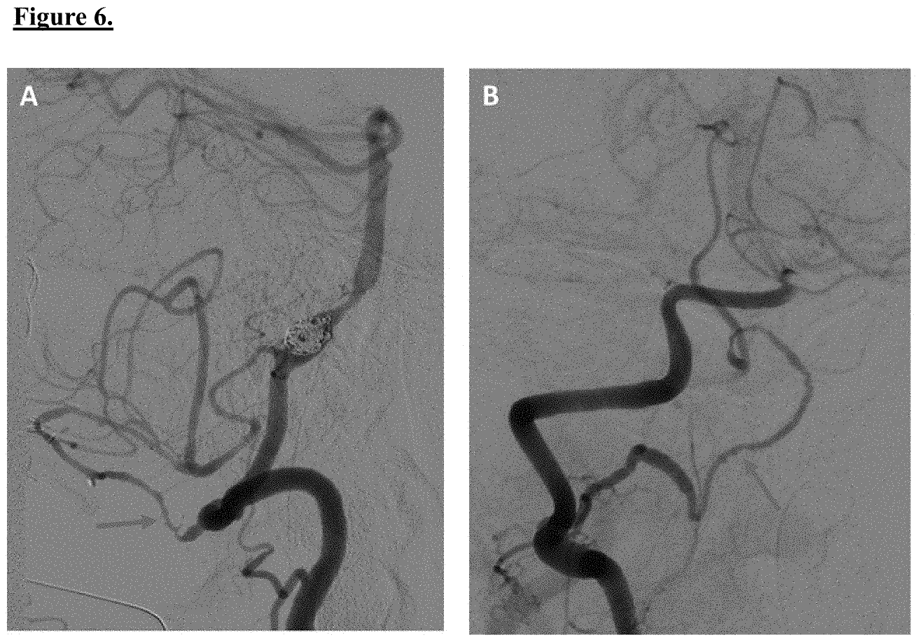

Thus, the present series represents the first clinical use of BTX for the prevention of arterial graft spasm and provides preliminary evidence for the safety and efficacy of this approach. The impetus for BTX use came from previous cases of severe spasm with DLCFA grafts at our institution, despite a meticulous surgical technique and postoperative blood flow optimization (FIG. 6). Following the ex vivo application of BTX A prior to DLCFA graft implantation in the featured cases, none of the patients exhibited either clinical or radiographic evidence of graft spasm over the short-term to midterm follow-up. As most severe arterial graft spasms occur within the first few days after implantation, delayed spasm in these patients is unlikely. Botulinum toxin A was chosen for application given its extensive clinical safety profile, and histological analysis of the treated arteries revealed no short-term adverse effects on the endothelium or vessel wall. All 3 subjects described herein also had uneventful postoperative and outpatient courses. These data support the utility of BTX treatment for the prevention of arterial graft spasm for cerebral and other bypass applications.

Ex vivo BTX treatment represents an appealing method for the long-term prevention of bypass graft spasm. These cases have not revealed any clinical safety concerns, and radiographic appearances have been markedly improved compared with prior clinical experience. Further data are needed to elucidate the role of BTX treatment in revascularization procedures.

### Example 7

### Further Studies

In follow-up studies, the Inventors applied botulinum toxin in 34 bypass procedures, with the describe technique widely applicable to a variety of indications. Indications were as follows, 16 for aneurysm, 10 for atherosclerotic vessel occlusion, 6 for moyamoya angiopathy, 1 for a traumatic vessel dissection and 1 for a tumor with major intracranial vessel involvement.

The Inventors harvested the descending branch of the lateral circumflex femoral artery (DLCFA) as a graft artery in 22 cases, the posterior tibial artery in 2 cases, a radial artery flow-through flap (RAFF) in 2 cases and a radial artery graft (RAG) in 8 cases. In all instances, the Inventors observed no radiographic or clinical graft spasm appreciated in any of these cases.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are sources of grafts, transplant techniques associated therein, additives including for example, botulinum toxin, disease and/or conditions treated by the aforementioned composition and methods, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method, comprising:
incubating a vascular graft with an anti-spasm agent;
transplanting the incubated vascular graft into a subject.

2. The method of claim 1, wherein the anti-spasm agent is botulinum toxin (BTX).

3. The method of claim 2, wherein the BTX is type A, B, C, D, E, F, G and combinations thereof.

4. The method of claim 1, wherein the vascular graft is an autograft.

5. The method of claim 1, wherein the vascular graft is an allograft.

6. The method of claim 1, wherein the vascular graft is an artery or vein.

7. The method of claim 6, wherein the artery is a radial artery or descending branch of the lateral circumflex femoral artery.

8. The method of claim 6, wherein the vein is a saphenous vein.

9. The method of claim 1, wherein transplanting the incubated vascular graft is for extracranial-intracranial bypass surgery.

10. The method of claim 1, wherein the subject is administered a vasodilator.

11. A method of bypass surgery, comprising:
transplanting a vascular graft into a subject, wherein the vascular graft has been treated with an anti-spasm agent.

12. The method of claim 11, wherein the anti-spasm agent is botulinum toxin (BTX).

13. The method of claim 12, wherein the BTX is type A, B, C, D, E, F, G and combinations thereof.

14. The method of claim 12, wherein treatment with BTX comprises incubation for about 5 to 120 mins.

15. The method of claim 14, wherein treatment with BTX comprises 5-250 U in 5-30 ml of saline.

16. The method of claim 11, wherein the bypass surgery is extracranial-intracranial bypass surgery.

17. The method of claim 16, wherein the vascular graft is a radial artery, descending branch of the lateral circumflex femoral artery or saphenous vein.

18. A method of preparing a vascular graft, comprising:
incubating a vascular graft with botulinum toxin (BTX).

19. The method of claim 18, wherein BTX is type A, B, C, D, E, F, G and combinations thereof.

20. The method of claim 18, wherein incubating the vascular graft with BTX is for about 5 to 120 mins.

21. The method of claim 20, wherein incubating the vascular graft with BTX comprises 5-250 U of BTX in 5-30 ml of saline.

22. A vascular graft made by the method of claim 18.
